(19) United States
(12) Patent Application Publication
(10) Pub. No.: US 2005/0241643 A1
(43) Pub. Date: Nov. 3, 2005

(54) Tracheostomy suctioning device a utility medical "double" apparatus

(76) Inventor: Carolyn Murphy Webber, Shelby, NC (US)

Correspondence Address: MRS. CAROLYN MURPHY-WEBBER, 412 WEST GRAHAM STREET, SHELBY, NC, 28150, US

(21) Appl. No.: 11/108,167

(22) Filed: Apr. 16, 2005

Related U.S. Application Data
(60) Provisional application No. 60/563,414, filed on Apr. 19, 2004.

(51) Int. Cl. A62B 9/06; A61M 16/00

(52) U.S. Cl. 128/207.14

(57) ABSTRACT

The “Webber-Y” is a hand-held suctioning device with dual functionality that allows a tracheostomy patient or healthcare worker to efficiently suction secretions from the tracheostomy tube area and from the mouth of the patient in a sanitary manner. It is a disposable device that is transportable and features color-coded end caps allowing the patient the freedom to be mobile and to preserve his/her dignity as a tracheostomized patient.

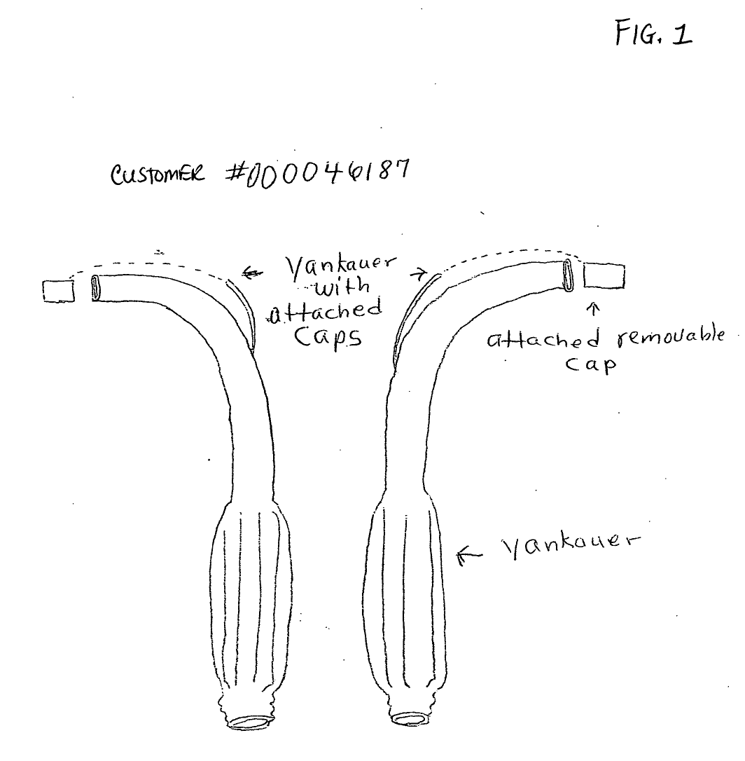

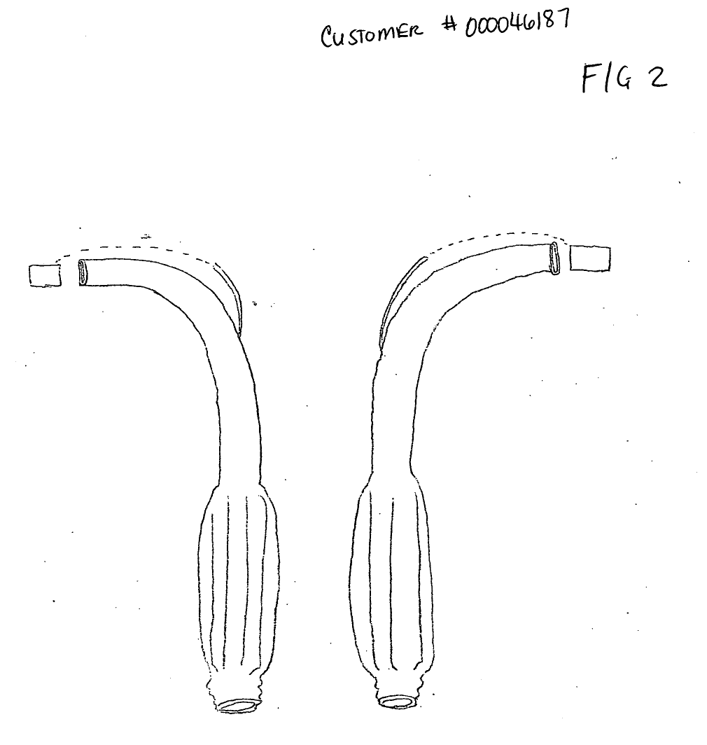

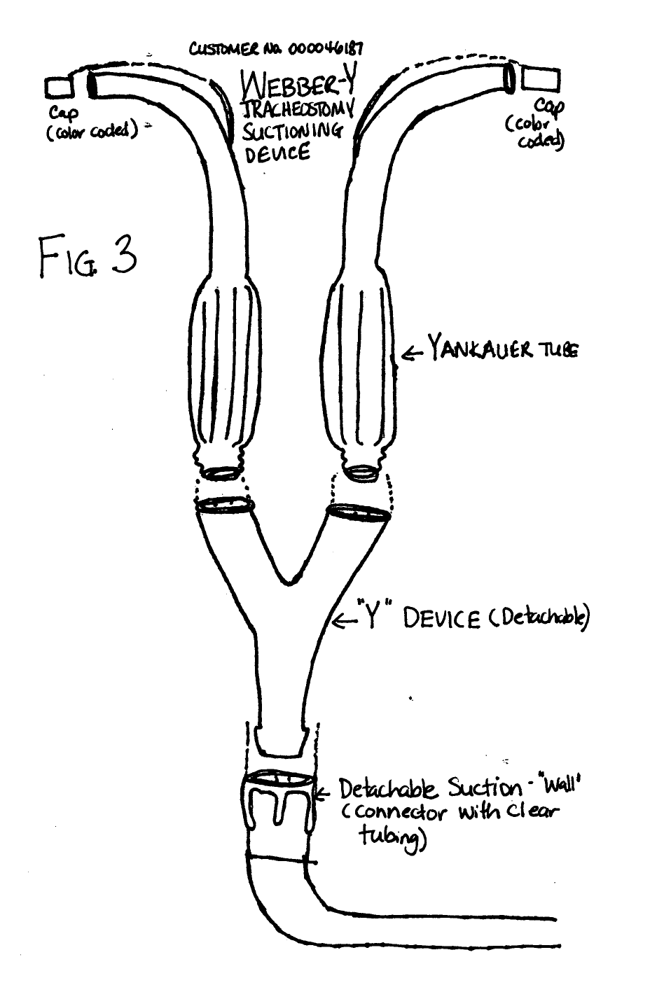

The invention relates to improving existing suctioning devices by combining two devices on a single control. This control is the “Y” (pronounced Wi) as in the alphabet. One side of the device is for the mouth secretions, the other is for the mucus secretions from the tracheostomy end protruding from the patients neck. Color-coded caps are attached on each Yaunker tube. The caps control the suction flow using one Yaunker tube at a time.

### CLAIMS/METHODS/DETAILED DESCRIPTION OF INVENTION

Patients and healthcare workers will like using the “Webber-Y” because it makes the patient feel more comfortable. And if the patient is physically capable, it will allow him/her more freedom and independence from having to wait for healthcare workers to provide relief from secretion build-up. And it will also provide the patient a sense of dignity. The device is always connected, ready for use on the bed. It is great to use between deep-suctioning periods. There are portable machines that the “Webber-Y” can be attached at home or traveling. Using the machine can speed recovery time. It is desired to design The “Webber-Y” where as it has dual functions on a “single” control. It will be a disposable apparatus.

### SUMMARY OF INVENTION

The invention relates to improving existing suctioning devices by combining two devices on a single control. One side of the device is for the mouth secretions, the other is for the mucus secretions from the tracheostomy end protruding from the patients neck. Color-coded caps are attached on each Yaunker tube. The caps control the suction flow using one Yaunker tube at a time. The “Webber-Y” where as it has dual functions on a “single” control. It will be a disposable apparatus.

1. Tracheostomized patients and healthcare workers who care for tracheostomized patients will like using the “Webber-Y” because it can make the patient feel more comfortable by using the “Webber-Y” to efficiently remove secretions expelled from the lungs into the mouth and into the tracheostomy tube from either deep suctioning and/or during coughing action.

2. If the patient is physically capable, use of the device of claim 1 will allow him/her more freedom and independence from having to wait for healthcare workers to provide relief from secretion build-up in the mouth and in the tracheostomy tube.

3. And the use of the device of claim 1 will also provide the patient a sense of dignity; the device can always be connected and ready for use.

4. The use of the device of claim 1 is great to use between deep-suctioning periods, as there are portable machines to which the “Webber-Y” can be attached at home or traveling and the device should be made of plastic material allowing for quick changing of the plastic suction tubes (Yankauer), when necessary.

5. Using the device of claim 1 can speed recovery time.

6. It is desired to design the “Webber-Y” whereas it has dual functions on a “single control and features color-coded, removable end caps to distinguish between the side of the “Y” tube for use to clear the mouth from side used to clear the tracheostomy area to help ensure good hygiene is maintained.

7. The Webber-Y, use of the device of claim 1, will be a disposable apparatus as to help ensure good hygiene.
